(19) United States
(12) Patent Application Publication
(10) Pub. No.: US 2024/0341746 A1
(43) Pub. Date: Oct. 17, 2024

(54) Variable Angle Button-Suture Fixation Assembly and Related Methods

(71) Applicant: Dunamis Medical Technologies, LLC, Greenville, AL (US)

(72) Inventor: Prithviraj Chavan, Greenville, AL (US)

(21) Appl. No.: 18/634,816

(22) Filed: Apr. 12, 2024

Related U.S. Application Data
(60) Provisional application No. 63/458,925, filed on Apr. 12, 2023.

(51) Int. Cl. A61B 17/04

(52) U.S. Cl. CPC A61B 17/0401; A61B 2017/0404; A61B 2017/0446; A61B 2017/0464

(57) ABSTRACT

Variable angle button-suture fixation assemblies (or “variable angle button assemblies” or “tensionable fixation assemblies”) that enable suture assemblies to be deployed at more extreme angles, which are sometimes necessitated by the affected patient anatomy. The devices disclosed herein allow for the user to adjust the tension in a tensionable fixation member (e.g., surgical suture) at a more extreme angle than allowed by other systems (and also to adjust the angle of repair if needed during use), and then lock the construct with the tensionable fixation member under tension at the adjusted angle. Although described herein in the context of a knotless repair technique, the variable angle button-suture fixation assemblies described herein may be configured for use in a knotted fixation technique, or an interlocking suture fixation technique.

### CROSS-REFERENCES TO RELATED APPLICATIONS

The present application is a nonprovisional application claiming the benefit of priority under 35 U.S.C. § 119 (e) from U.S. Provisional Application No. 63/458,925, filed on Apr. 12, 2023, the complete disclosure of which is hereby incorporated by reference into this disclosure as if set forth fully herein.

### FIELD

The present disclosure relates generally to soft tissue repair reconstruction, and more particularly to devices which will allow for soft tissue, bone, allograft, tendon, and/or graft tension members to be fixed at variable angles relative to the fixation device.

### BACKGROUND

Tendon, ligament, and joint capsular injuries account for 45 percent of orthopedic injuries who would seek medical attention. Tendon injuries alone account for 30 million people annually which results in enormous amount of financial and physical burden to both the individual and the economy. Most devices used for repair of these injuries require knot-tying or knotless fixation for securing soft tissue to bone or bone to bone. Traditionally, when a fixation is performed, for fractures or soft tissue repair or bone to bone repair indications, if a pilot hole is made through the bone to assist with fixation with an anchoring device, the fixation is uniplanar. The pilot hole essentially defining the plane of fixation or if a plate or washer is used to assist in fracture repair or soft tissue repair the pilot hole can only be drilled in a single plane of the device to completely sit within the plate. This may limit the strength of repair because there may be instances in which a tensioned repair is needed at more extreme angles.

### SUMMARY

The present disclosure describes a variable angle fixation assembly that can be used to perform fixation with or without a plating system. It can allow the surgeons to drill at variable angles. The user of the assembly can still perform the locking fixation and variable angles. This assembly allows for two congruent articulating surfaces which will permit variable angle fixation. The assembly and method of repair disclosed herein utilizes a button device along with tensionable fixation members (e.g., surgical suture) that can assist with performing this repair. The device disclosed herein may be used for various soft tissue to bone and bone to bone applications. The device disclosed herein may be used as an adjunct for fracture fixation. Some of the examples of applications include syndesmosis repair, AC repair, ACL repair, PCL repair, Fibula fractures, etc.

In some embodiments, the variable angle button-suture fixation assembly and method of repair disclosed herein allows for multiplanar fixation.

In some embodiments, the variable angle button-suture fixation assembly and method of repair disclosed herein allows for drilling in different angles through a plate or a washer which can still stay flush on the bone while drilling.

In some embodiments, the variable angle button-suture fixation assembly and method of repair disclosed herein creates a low-profile fixation option.

In some embodiments, the variable angle button-suture fixation assembly and method of repair disclosed herein reduces the risk of revisions related to hardware prominence.

In some embodiments, the variable angle button-suture fixation assembly and method of repair disclosed herein interacts with an articulating surface of a fixation plate.

In some embodiments, the variable angle button-suture fixation assembly and method of repair disclosed herein may be performed in a knotless manner using the locking interface of the device/construct to achieve locking of the construct.

In some embodiments, the variable angle button-suture fixation assembly and method of repair disclosed herein may be performed using an interwoven suture pattern (e.g., “finger trap design”) to achieve locking of the construct.

In some embodiments, the variable angle button-suture fixation assembly and method of repair disclosed herein may be performed using a knot-tying method to achieve locking of the construct.

Described herein are several embodiments of a variable angle button-suture fixation assembly (or “variable angle button assembly” or “tensionable fixation assembly”) that enable suture assemblies to be deployed at more extreme angles, which are sometimes necessitated by the affected patient anatomy. The devices disclosed herein allow for the user to adjust the tension in a tensionable fixation member (e.g., surgical suture) at a more extreme angle than allowed by other systems (and also to adjust the angle of repair if needed during use), and then lock the construct with the tensionable fixation member under tension at the adjusted angle. Although described herein in the context of a knotless repair technique, the variable angle button-suture fixation assemblies described herein may be configured for use in a knotted fixation technique, or an interlocking suture fixation technique. By way of example, the knotless button-suture fixation technique referred to herein is generally shown and described in commonly-owned U.S. Pat. No. 11,109,855, issued Sep. 7, 2021, and entitled “Knotless Orthopedic Stabilization System” (“the '855 patent”), the entire contents of which is incorporated by reference as if set forth fully herein. Although shown and described herein as a surgical suture, it should be understood that the tensionable fixation member may be any form of flexible element capable of being locked under tension, including but not limited to surgical sutures, tapes, filaments, threads, wires, etc.

In some embodiments, the variable angle button assembly includes a button, a locking element, and a receiver. By way of example, the button is received within an opening or through-hole in the receiver, which for example may be an orthopedic fixation plate, another button, or a prosthesis, and the locking element is received within an opening or through-hole in the button. In this example embodiment, the opening in the receiver includes an inner recess configured to receive the button therein in such a way that the button does not move relative to the receiver. In some embodiments, the receiver further includes a chamfered, rounded, smooth, or angled surface at the distal rim of the opening to minimize the stress on the tensionable fixation member (e.g., surgical suture) as it passes through and potentially contacts the receiver during use, while enabling more extreme fixation angles.

In some embodiments, the button has an outer circumferential surface having a size and shape complementary to the size and shape of the inner recess to facilitate the non-moving engagement between the button and receiver. In some embodiments, the button further includes an inner circumferential surface configured to interface with the outer circumferential surface of the locking element. In some embodiments, the inner circumferential surface of the button is concave and the outer circumferential surface of the locking element is convex, such that the locking element is angularly moveable when seated within the button. In some embodiments, the button may have an upper or proximal surface that is chamfered to reduce or eliminate prominence of the button beyond the proximal surface of the receiver.

In some embodiments, the locking element further includes a distal protrusion having one or more transverse openings configured to receive one or more loops of a tensionable fixation member therethrough. In some embodiments, the locking element may have an upper or proximal surface that is chamfered to reduce or eliminate prominence of the locking element beyond the proximal surface of the receiver.

In use, a portion of a tensionable fixation member (e.g., one or more distal loop portions) is attached to or coupled with a tissue, bone, or other element while at least one other portion of the tensionable fixation member (e.g., proximal loop portion) is looped through the transverse opening. One or more free ends of the tensionable fixation member pass between the button and the locking element within one or more lateral indentations formed within the outer circumferential surface of the locking element. Prior to final tightening of the construct, the angle of the locking element relative to the button (and receiver) may be adjusted to a desired or optimal degree to maintain tension in the tensionable fixation member. In some embodiments, the construct can then be locked with the desired tension in the tensionable fixation member by exerting a proximal force (e.g., pulling) on the free ends of the tensionable fixation member. This creates “pinch points” which capture and lock the tensionable fixation member between the button and locking element within the lateral indentations while maintaining the adjusted angle of the locking element. In some embodiments, the construct can be locked while maintaining the adjusted angle of the locking element by tying one or more knots with the free ends or by interweaving the free ends in a finger-trap type design.

In some embodiments, the variable angle button assembly includes a button, a locking element, and a receiver. By way of example, the button is received within an opening or through-hole in the receiver, which for example may be an orthopedic fixation plate, and the locking element is received within a recess formed in a proximal surface of the button. In this example embodiment, the opening in the receiver includes an inner recess configured to receive the button therein in such a way that the button is angularly moveable relative to the receiver when the button is seated within the inner recess. In some embodiments, the inner recess comprises a concave circumferential surface configured to interface with a convex outer circumferential surface of the button such that the button is angularly moveable when seated within the recess. In some embodiments, the receiver further includes a chamfered, rounded, smooth, or angled surface at the distal rim of the opening to minimize the stress on the tensionable fixation member (e.g., surgical suture) as it passes through and potentially contacts the receiver during use, while enabling more extreme fixation angles.

In some embodiments, the button includes an inner circumferential surface configured to interface with the outer circumferential surface of the locking element such that the locking element does not angularly move relative to the button. In some embodiments, the inner circumferential surface of the button is conical and the outer circumferential surface of the locking element is also conical with a slope that matches the slope of the inner circumferential surface, such that the locking element is angularly static when seated within the button. In some embodiments, the inner circumferential surface of the button and outer circumferential surface of the locking element may have complimentary surface structures (e.g., protrusion and recess) that facilitate a keyed engagement between the locking element and button. In any event, the button has an inner circumferential surface having a size and shape complementary to the size and shape of the outer circumferential surface of the locking element to facilitate the non-moving engagement between the button and locking element. In some embodiments, the locking element further includes a distal protrusion having one or more transverse openings configured to receive one or more loops of a tensionable fixation member therethrough.

In some embodiments, the button includes a central opening formed within the recess and sized and configured to receive at least a portion of the distal protrusion therethrough while preventing angular movement of the locking element while the distal protrusion is inserted into the central opening. In some embodiments, the button includes one or more lateral openings positioned within the recess on either side of the central opening and configured to allow passage of the tensionable fixation member therethrough. In some embodiments, the central opening may be formed perpendicular to a plane defined by the distal (e.g., bone or tissue engaging) surface of the receiver.

In some embodiments, the central opening may be formed at an oblique angle relative to a plane defined by the distal (e.g., bone or tissue engaging) surface of the receiver. By way of example, this may include embodiments in which neither the button nor the locking element is angularly moveable relative to each other or the receiver. Thus, more extreme fixation angles may be achieved by providing a button with the oblique angled central opening and a locking element with a complimentary oblique angled protrusion. While not a variable angle construct per se, a surgical kit may be provided with buttons and locking elements having several different fixation angles, e.g., 20°, 25°, 30°, 35° 40°, 45°, 50°, 55°, 60°, etc., to accommodate the needs of the individual surgeon at the time of surgery.

In some embodiments, the button may have an upper or proximal surface that is chamfered to reduce or eliminate prominence of the button beyond the proximal surface of the receiver. In some embodiments, the locking element may have an upper or proximal surface that is chamfered to reduce or eliminate prominence of the locking element beyond the proximal surface of the receiver.

In use, a portion of a tensionable fixation member (e.g., one or more distal loop portions) is attached to or coupled with a tissue, bone, or other element while at least one other portion (e.g., one or more proximal loop portions) of the tensionable fixation member is looped through the transverse opening. One or more free ends of the tensionable fixation member pass between the button and the locking element within one or more lateral indentations formed within the outer circumferential surface of the locking element. Prior to final tightening of the construct, the angle of the button relative to the receiver may be adjusted to a desired or optimal degree to maintain tension in the tensionable fixation member. In some embodiments, the construct can then be locked with the desired tension in the tensionable fixation member by exerting a proximal force (e.g., pulling) on the free ends of the suture. This creates “pinch points” which capture and lock the tensionable fixation member between the button and locking element within the lateral indentations while maintaining the adjusted angle of the button relative to the receiver. In some embodiments, the construct can be locked while maintaining the adjusted angle of the button by tying one or more knots with the free ends or by interweaving the free ends in a finger-trap type design.

In some embodiments, the variable angle button assembly does not include a separate locking element with a protrusion, which allows for even more extreme fixation angles. Instead, locking of the tensionable fixation member occurs via interface between the button and the receiver, as will be explained below. In some embodiments, the variable angle button assembly includes a button and a receiver. By way of example, the button is received within an opening or through-hole in the receiver, which for example may be an orthopedic fixation plate. In this example embodiment, the opening in the receiver includes an inner recess configured to receive the button therein in such a way that the button is angularly moveable relative to the receiver when the button is seated within the inner recess. In some embodiments, the inner recess comprises a concave circumferential surface configured to interface with a convex outer circumferential surface of the button such that the button is angularly moveable when seated within the recess. In some embodiments, the receiver further includes a chamfered, rounded, smooth, or angled surface at the distal rim of the opening to minimize the stress on the tensionable fixation member (e.g., surgical suture) as it passes through and potentially contacts the receiver during use, while enabling more extreme fixation angles.

In some embodiments, the button is round in shape (or oval, elliptical, or any geometric shape that allows for variable angle fixation) and includes a pair of lateral openings positioned on either side of a central crossbar and a pair of opposing lateral indentations formed within the outer circumferential surface. The central crossbar and lateral openings enable a portion of the tensionable fixation member to be looped over the crossbar during use. Although shown herein as having a pair of lateral openings, the button may be configured with more or less than two lateral openings without departing from the scope of the disclosure.

In use, a portion of a tensionable fixation member (e.g., a distal loop portion) is attached to or coupled with a tissue, bone, or other element while at least one other portion (e.g., a proximal loop portion) of the tensionable fixation member is looped around the central crossbar by way of the lateral openings. One or more free ends of the tensionable fixation member pass between the button and the receiver within the one or more lateral indentations formed within the outer circumferential surface of the button. Prior to final tightening of the construct, the angle of the button relative to the receiver may be adjusted to a desired or optimal degree to maintain tension in the tensionable fixation member. In some embodiments, the construct can then be locked with the desired tension in the tensionable fixation member by exerting a proximal force (e.g., pulling) on the free ends of the tensionable fixation member. This creates “pinch points” which capture and lock the tensionable fixation member between the button and receiver within the lateral indentations while maintaining the adjusted angle of the button relative to the receiver. In some embodiments, the construct can be locked while maintaining the adjusted angle of the button by tying one or more knots with the free ends or by interweaving the free ends in a finger-trap type design.

In some embodiments, the variable angle button assembly includes a button and a receiver. By way of example, the button is received within an opening or through-hole in the receiver, which for example may be an orthopedic fixation plate. In this example embodiment, the opening in the receiver includes an inner recess configured to receive the button therein in such a way that the button is angularly moveable relative to the receiver when the button is seated within the inner recess. In some embodiments, the inner recess comprises a concave circumferential surface configured to interface with a convex outer circumferential surface of the button such that the button is angularly moveable when seated within the recess. In some embodiments, the receiver further includes a chamfered, rounded, smooth, or angled surface at the distal rim of the opening to minimize the stress on the tensionable fixation member (e.g., surgical suture) as it passes through and potentially contacts the receiver during use, while enabling more extreme fixation angles.

In some embodiments, the button is round in shape (or oval, elliptical, or any geometric shape that allows for variable angle fixation) and includes a pair of lateral openings positioned on either side of a central crossbar and a pair of opposing lateral indentations formed within the outer circumferential surface. The central crossbar and lateral openings enable a portion of the tensionable fixation member to be looped over the crossbar during use. Although shown herein as having a pair of lateral openings, the button may be configured with more or less than two lateral openings without departing from the scope of the disclosure. In some embodiments, the proximal side of the button may include angle surfaces or chamfers to minimize or eliminate any portion of the button that may otherwise protrude beyond the plane of the top or proximal surface of the receiver, when the button is oriented at an angle relative to the receiver.

In use, a portion of a tensionable fixation member (e.g., a distal loop portion) is attached to or coupled with a tissue, bone, or other element while at least one other portion of the tensionable fixation member (e.g., a proximal loop portion) is looped around the central crossbar by way of the lateral openings. One or more free ends of the tensionable fixation member pass between the button and the receiver within the one or more lateral indentations formed within the outer circumferential surface of the button. Prior to final tightening of the construct, the angle of the button relative to the receiver may be adjusted to a desired or optimal degree to maintain tension in the tensionable fixation member. In some embodiments, the construct can then be locked with the desired tension in the tensionable fixation member by exerting a proximal force (e.g., pulling) on the free ends of the suture. This creates “pinch points” which capture and lock the tensionable fixation member between the button and receiver within the lateral indentations while maintaining the adjusted angle of the button relative to the receiver. In some embodiments, the construct can be locked while maintaining the adjusted angle of the button by tying one or more knots with the free ends or by interweaving the free ends in a finger-trap type design.

In some embodiments, the variable angle button assembly includes a button, a locking element, and a receiver. By way of example, the button is received within an opening or through-hole in the receiver, which for example may be an orthopedic fixation plate, and the locking element is received within an opening or through-hole in the button. In this example embodiment, the opening in the receiver includes an inner recess configured to receive the button therein in such a way that the button does not move relative to the receiver. In some embodiments, the receiver further includes a chamfered, rounded, smooth, or angled surface at the distal rim of the opening to minimize the stress on the tensionable fixation member (e.g., surgical suture) as it passes through and potentially contacts the receiver during use, while enabling more extreme fixation angles.

In some embodiments, the button has an outer circumferential surface having a size and shape complementary to the size and shape of the inner recess to facilitate the non-moving engagement between the button and receiver. In some embodiments, the button further includes an inner circumferential surface configured to interface with the outer circumferential surface of the locking element. In some embodiments, the inner circumferential surface of the button is concave and the outer circumferential surface of the locking element is convex, such that the locking element is angularly moveable when seated within the button. In some embodiments, the button may have an upper or proximal surface that is chamfered to reduce or eliminate prominence of the button beyond the proximal surface of the receiver.

In some embodiments, the locking element is round in shape (or oval, elliptical, or any geometric shape that allows for variable angle fixation) and includes a pair of lateral openings positioned on either side of a central crossbar and a pair of opposing lateral indentations formed within the outer circumferential surface. The central crossbar and lateral openings enable a portion of the tensionable fixation member to be looped over the crossbar during use. Although shown herein as having a pair of lateral openings, the locking element may be configured with more or less than two lateral openings without departing from the scope of the disclosure. In some embodiments, the locking element may have an upper or proximal surface that is chamfered to reduce or eliminate prominence of the locking element beyond the proximal surface of the receiver.

In use, a portion of a tensionable fixation member (e.g., a distal loop portion) is attached to or coupled with a tissue, bone, or other element while at least one other portion (e.g., a proximal loop portion) of the tensionable fixation member is looped around the central crossbar by way of the lateral openings. One or more free ends of the tensionable fixation member pass between the button and the locking element within one or more lateral indentations formed within the outer circumferential surface of the locking element. Prior to final tightening of the construct, the angle of the locking element relative to the button (and receiver) may be adjusted to a desired or optimal degree to maintain tension in the tensionable fixation member. In some embodiments the construct can then be locked with the desired tension in the tensionable fixation member by exerting a proximal force (e.g., pulling) on the free ends of the tensionable fixation member. This creates “pinch points” which capture and lock the tensionable fixation member between the button and locking element within the lateral indentations while maintaining the adjusted angle of the locking element. In some embodiments, the construct can be locked while maintaining the adjusted angle of the locking element by tying one or more knots with the free ends or by interweaving the free ends in a finger-trap type design.

In some embodiments, the variable angle button assembly includes a button, a locking element, and a receiver. By way of example, the button is received within an opening or through-hole in the receiver, which for example may be an orthopedic fixation plate, and the locking element is received within a recess formed in a proximal surface of the button. In this example embodiment, the opening in the receiver includes an inner recess configured to receive the button therein in such a way that the button is angularly moveable relative to the receiver when the button is seated within the inner recess. In some embodiments, the inner recess comprises a concave circumferential surface configured to interface with a convex outer circumferential surface of the button such that the button is angularly moveable when seated within the recess. In some embodiments, the receiver further includes a chamfered, rounded, smooth, or angled surface at the distal rim of the opening to minimize the stress on the tensionable fixation member (e.g., surgical suture) as it passes through and potentially contacts the receiver during use, while enabling more extreme fixation angles.

In some embodiments, the button includes an inner circumferential surface configured to interface with the outer circumferential surface of the locking element such that the locking element does not angularly move relative to the button. In some embodiments, the inner circumferential surface of the button is conical and the outer circumferential surface of the locking element is also conical with a slope that matches the slope of the inner circumferential surface, such that the locking element is angularly static when seated within the button. In some embodiments, the inner circumferential surface of the button and outer circumferential surface of the locking element may have complimentary surface structures (e.g., protrusion and recess) that facilitate a keyed engagement between the locking element and button. In any event, the button has an inner circumferential surface having a size and shape complementary to the size and shape of the outer circumferential surface of the locking element to facilitate the non-moving engagement between the button and locking element. In some embodiments, the button may have an upper or proximal surface that is chamfered to reduce or eliminate prominence of the button beyond the proximal surface of the receiver.

In some embodiments, the locking element is round in shape (or oval, elliptical, or any geometric shape that allows for variable angle fixation) and includes a pair of lateral openings positioned on either side of a central crossbar and a pair of opposing lateral indentations formed within the outer circumferential surface. The central crossbar and lateral openings enable a portion of the tensionable fixation member to be looped over the crossbar during use. Although shown herein as having a pair of lateral openings, the locking element may be configured with more or less than two lateral openings without departing from the scope of the disclosure. In some embodiments, the locking element may have an upper or proximal surface that is chamfered to reduce or eliminate prominence of the locking element beyond the proximal surface of the receiver.

In use, a portion of a tensionable fixation member (e.g., a distal loop portion) is attached to or coupled with a tissue, bone, or other element while at least one other portion (e.g., a proximal loop portion) of the tensionable fixation member is looped around the central crossbar by way of the lateral openings. One or more free ends of the tensionable fixation member pass between the button and the locking element within one or more lateral indentations formed within the outer circumferential surface of the locking element. Prior to final tightening of the construct, the angle of the button relative to the receiver may be adjusted to a desired or optimal degree to maintain tension in the tensionable fixation member. In some embodiments, the construct can then be locked with the desired tension in the tensionable fixation member by exerting a proximal force (e.g., pulling) on the free ends of the suture. This creates “pinch points” which capture and lock the tensionable fixation member between the button and locking element within the lateral indentations while maintaining the adjusted angle of the button relative to the receiver. In some embodiments, the construct can be locked while maintaining the adjusted angle of the button by tying one or more knots with the free ends or by interweaving the free ends in a finger-trap type design.

In some embodiments, the variable angle button assembly includes a button, a locking element, and a receiver. By way of example, the button is received within an opening or through-hole in the receiver, which for example may be an orthopedic fixation plate, and the locking element is received within a recess formed in a proximal surface of the button. In this example embodiment, the opening in the receiver includes an inner recess configured to receive the button therein in such a way that the button is angularly moveable relative to the receiver when the button is seated within the inner recess. In some embodiments, the inner recess comprises a concave circumferential surface configured to interface with a convex outer circumferential surface of the button such that the button is angularly moveable when seated within the recess. In some embodiments, the receiver further includes a chamfered, rounded, smooth, or angled surface at the distal rim of the opening to minimize the stress on the tensionable fixation member (e.g., surgical suture) as it passes through and potentially contacts the receiver during use, while enabling more extreme fixation angles.

In some embodiments, the button includes a central opening formed within the recess and sized and configured to enable passage of the tensionable fixation member or other flexible fixation member therethrough. In some embodiments, the button includes one or more lateral openings positioned within the recess on either side of the central opening and configured to allow passage of the tensionable fixation member therethrough. Although shown herein as having a pair of lateral openings, the button may be configured with more or less than two lateral openings without departing from the scope of the disclosure.

In some embodiments, the button includes an inner circumferential surface configured to interface with the outer circumferential surface of the locking element such that the locking element does not angularly move relative to the button. In some embodiments, the inner circumferential surface of the button is conical and the outer circumferential surface of the locking element is also conical with a slope that matches the slope of the inner circumferential surface, such that the locking element is angularly static when seated within the button. In some embodiments, the inner circumferential surface of the button and outer circumferential surface of the locking element may have complimentary surface structures (e.g., protrusion and recess) that facilitate a keyed engagement between the locking element and button. In any event, the button has an inner circumferential surface having a size and shape complementary to the size and shape of the outer circumferential surface of the locking element to facilitate the non-moving engagement between the button and locking element. In some embodiments, the button may have an upper or proximal surface that is chamfered to reduce or eliminate prominence of the button 612 beyond the proximal surface of the receiver.

In some embodiments, the locking element is round in shape (or oval, elliptical, or any geometric shape that allows for variable angle fixation) and includes a pair of lateral openings positioned on either side of a central crossbar and a pair of opposing lateral indentations formed within the outer circumferential surface. The central crossbar and lateral openings enable a portion of the tensionable fixation member to be looped over the crossbar during use. Although shown herein as having a pair of lateral openings, the locking element may be configured with more or less than two lateral openings without departing from the scope of the disclosure. In some embodiments, the locking element may have an upper or proximal surface that is chamfered to reduce or eliminate prominence of the locking element beyond the proximal surface of the receiver.

In use, a portion of a tensionable fixation member (e.g., a distal loop portion) is attached to or coupled with a tissue, bone, or other element while at least one other portion (e.g., a proximal loop portion) of the tensionable fixation member is looped around the central crossbar by way of the lateral openings of the locking element and the central opening of the button. One or more free ends of the tensionable fixation member pass through lateral openings of the button and then pass between the button and the locking element within one or more lateral indentations formed within the outer circumferential surface of the locking element. Prior to final tightening of the construct, the angle of the button relative to the receiver may be adjusted to a desired or optimal degree to maintain tension in the tensionable fixation member. In some embodiments, the construct can then be locked with the desired tension in the tensionable fixation member by exerting a proximal force (e.g., pulling) on the free ends of the suture. This creates “pinch points” which capture and lock the tensionable fixation member between the button and locking element within the lateral indentations while maintaining the adjusted angle of the button relative to the receiver. In some embodiments, the construct can be locked while maintaining the adjusted angle of the button by tying one or more knots with the free ends or by interweaving the free ends in a finger-trap type design.

Although shown and described herein as a fixation plate, it should be understood that the receivers described herein may be any device that is capable or for has been adapted to receive the button therein, including but not limited to a fixation plate, button, prosthesis, etc. Moreover, the receiver may include multiple openings for use with multiple variable angle buttons and/or non variable angle buttons and/or bone screws to achieve fixation. In some embodiments, the device can be used without a receiver member, or stated another way, the receiver and button may be interchangeable elements such that the device may be placed directly on the bone without a fixation plate or other intermediary device.

By way of example, the buttons and/or locking elements described herein may be adjusted to any angle (e.g., “adjustable angle” or “fixation angle” measured between a longitudinal axis of the opening or through-hole of the receiver and a longitudinal axis of the button) up to a maximum angle that is allowable by the physical constraints of the receiver. By way of example, when the button is seated in the receiver in an angularly neutral position, the longitudinal axis of the button and the longitudinal axis of the receiver are in alignment, with 0° of angulation between them. As used herein, the term “angularly moveable” means that the button (prior to final tightening) may move (e.g., pivot, rotate, etc.) within the inner recess of the receiver from the “neutral angle” to an “adjusted angle” where the angle between the longitudinal axis of the receiver and the longitudinal axis of the button is nonzero.

Similarly, when the locking element is seated in the button, at an angularly neutral position, the longitudinal axis of the button and the longitudinal axis of the locking element are in alignment, with 0° of angulation between them. As used herein, the term “angularly moveable” means that the locking element (prior to final tightening) may move (e.g., pivot, rotate, etc.) within the inner recess of the button from the “neutral angle” to an “adjusted angle” where the angle between the longitudinal axis of the button and the longitudinal axis of the locking element is nonzero.

In some embodiments, the adjustable angle may be within a range between 1° to 85°, which results in a total angular range of between 5° and 175° relative to the plane defined by the distal surface of the receiver. In some embodiments, the adjustable angle may be within a range between 1° and 70°, which results in a total angular range between 20° and 160°. In some embodiments, the adjustable angle may be within a range between 1° and 60°, which results in a total angular range of 30-150°.

In some embodiments, the button, locking element, and/or receiver may be manufactured from any suitable surgical grade material, including but not limited to metal (e.g., titanium), plastics (e.g., PEEK), or other materials.

As additional description to the embodiments described below, the present disclosure describes the following embodiments.

Embodiment 1 is a tensionable fixation assembly configured to fix tissue or bone to tissue, bone, or other member, comprising: a button comprising a proximal facing surface, a distal facing surface, a circumferential outer mating surface, and at least one opening extending therethrough, the button defining a first end of the tensionable fixation assembly; a receiver having an opening extending therethrough and an inner recess surrounding the opening, the inner recess defining a receiving surface configured to interface with the button such that the button is angularly moveable relative to the receiver and configured to be secured within the recess at variable fixation angles while maintaining contact between the outer mating surface and the receiving surface; and at least one tensionable fixation member interacting with the button and receiver.

Embodiment 2 is the tensionable fixation assembly of embodiment 1, wherein the at least one tensionable fixation member has a proximal end portion extending proximally from the button and a first loop portion extending distally from the receiver and configured to mate with tissue, bone, or other member, the first loop portion defining a second end of the tensionable fixation assembly.

Embodiment 3 is the tensionable fixation assembly of embodiments 1 or 2, wherein the tensionable fixation assembly comprises a first state defining a first distance between the first and second ends and second state defining a second distance between the first and second ends, the second distance being less than the first distance.

Embodiment 4 is the tensionable fixation assembly of any of embodiments 1 through 3, wherein the at least one tensionable fixation member passes through the button and receiver.

Embodiment 5 is the tensionable fixation assembly of any of embodiments 1 through 4, wherein the at least one tensionable fixation member passes through the button and receiver by passing between the receiving surface of the receiver and the mating surface of the button.

Embodiment 6 is the tensionable fixation assembly of any of embodiments 1 through 5, wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained in a knotless manner via compression and friction applied to the tensionable fixation member between the receiving surface of the receiver and the mating surface of the button while maintaining the fixation angle of the button relative to the receiver.

Embodiment 7 is the tensionable fixation assembly of any of embodiments 1 through 6 wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained by tying one or more knots in the proximal end portion of the tensionable fixation member while maintaining the fixation angle of the button relative to the receiver.

Embodiment 8 is the tensionable fixation assembly of any of embodiments 1 through 7, wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained by interweaving the proximal end portion of the tensionable fixation member while maintaining the fixation angle of the button relative to the receiver.

Embodiment 9 is the tensionable fixation assembly of any of embodiments 1 through 8, wherein the interweaving comprises a finger trap design.

Embodiment 10 is the tensionable fixation assembly of any of embodiments 1 through 9, wherein the receiver comprises an orthopedic fixation plate, a second button, or a prosthesis.

Embodiment 11 is the tensionable fixation assembly of any of embodiments 1 through 10, wherein the button comprises at least one chamfered proximal surface.

Embodiment 12 is a tensionable fixation assembly configured to fix tissue or bone to tissue, bone, or other member, comprising: a suspension device defining a first end and comprising a button and a locking element separable from the button, the button having a proximal facing surface, a distal facing surface, a circumferential outer mating surface, a proximal recess including a receiving surface configured to mate with the locking element, and at least one opening extending therethrough, the locking element having a mating surface configured to interface with the receiving surface of the button; a receiver having an opening extending therethrough and an inner recess surrounding the opening, the inner recess defining a receiving surface configured to interface with the button such that the button is angularly moveable relative to the receiver and configured to be secured within the recess at variable fixation angles while maintaining contact between the outer mating surface and the receiving surface; at least one tensionable fixation member interacting with the suspension device, the at least one tensionable fixation member having a proximal end portion extending proximally from the suspension device, the at least one tensionable fixation member passing through the suspension device and forming a first loop portion extending distally from the suspension device, the first loop portion defining a second end configured to mate with tissue, bone, or other member, the at least one tensionable fixation member passing through the suspension device between the receiving surface of the button and the mating surface of the locking element; wherein the tensionable fixation assembly comprises a first state defining a first distance between the first and second ends and second state defining a second distance between the first and second ends, the second distance being less than the first distance; wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained via compression and friction applied to the tensionable fixation member between the receiving surface of the button and the mating surface of the locking pin while maintaining the fixation angle of the button relative to the receiver.

Embodiment 13 is the tensionable fixation assembly of embodiment 12, wherein the mating surface of the locking element and the receiving surface of the button are configured to interface with each other such that the locking element does not angularly move relative to the button.

Embodiment 14 is the tensionable fixation assembly of embodiment 12 or 13, wherein the receiving surface of the button is conical, and the mating surface of the locking element is conical.

Embodiment 15 is the tensionable fixation assembly of any of embodiments 12 through 14, wherein the inner recess of the receiver comprises a concave circumferential surface and the outer mating surface of the button comprises a convex outer circumferential surface.

Embodiment 16 is the tensionable fixation assembly of any of embodiments 12 through 15, wherein the receiver comprises an orthopedic fixation plate, a second button, or a prosthesis.

Embodiment 17 is the tensionable fixation assembly of any of embodiments 12 through 16, wherein the fixation angle is within the range of between 1° and 60°.

Embodiment 18 is the tensionable fixation assembly of any of embodiments 12 through 17, wherein the fixation angle is within the range of between 1° and 70°.

Embodiment 19 is the tensionable fixation assembly of any of embodiments 12 through 18, wherein the fixation angle is within the range of between 1° and 85°.

Embodiment 20 is a tensionable fixation assembly configured to fix tissue or bone to tissue, bone, or other member, comprising: a suspension device defining a first end and comprising a button and a locking element separable from the button, the button having a proximal facing surface, a distal facing surface, a circumferential outer mating surface, a proximal recess including a receiving surface configured to mate with the locking element, and at least one opening extending therethrough, the locking element having a mating surface configured to mate with the receiving surface of the button such that the locking element is angularly moveable relative to the button and configured to be secured within the proximal recess at variable fixation angles while maintaining contact between the mating surface of the locking element and the receiving surface of the button; a receiver having an opening extending therethrough and an inner recess surrounding the opening and configured to receive the button therein such that the button is not angularly moveable relative to the receiver when the button is seated within the inner recess; at least one tensionable fixation member interacting with the suspension device, the at least one tensionable fixation member having a proximal end portion extending proximally from the suspension device, the at least one tensionable fixation member passing through the suspension device and forming a first loop portion extending distally from the suspension device, the first loop portion defining a second end configured to mate with tissue, bone, or other member, the at least one tensionable fixation member passing through the suspension device between the receiving surface of the button and the mating surface of the locking element; wherein the tensionable fixation assembly comprises a first state defining a first distance between the first and second ends and second state defining a second distance between the first and second ends, the second distance being less than the first distance; wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained via compression and friction applied to the tensionable fixation member between the receiving surface of the button and the mating surface of the locking element while maintaining the fixation angle of the locking element relative to the button.

Embodiment 21 is the tensionable fixation assembly of embodiment 20, wherein the inner recess of the button comprises a concave circumferential surface and the outer mating surface of the locking element comprises a convex outer circumferential surface.

Embodiment 22 is the tensionable fixation assembly of embodiments 20 or 21, wherein the button comprises at least one chamfered proximal surface.

Embodiment 23 is the tensionable fixation assembly of any of embodiments 20 through 22, wherein the receiver comprises an orthopedic fixation plate, a second button, or a prosthesis.

Embodiment 24 is the tensionable fixation assembly of any of embodiments 20 through 23, wherein the fixation angle is within the range of between 1° and 60°.

Embodiment 25 is the tensionable fixation assembly of any of embodiments 20 through 24, wherein the fixation angle is within the range of between 1° and 70°.

Embodiment 26 is the tensionable fixation assembly of any of embodiments 20 through 25, wherein the fixation angle is within the range of between 1° and 85°.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a perspective view of an example of a variable angle button-suture fixation assembly, according to some embodiments;

FIG. 2 is a sectional view of the variable angle button-suture fixation assembly of FIG. 1, according to some embodiments;

FIG. 3 is an exploded perspective view of the variable angle button-suture fixation assembly of FIG. 1, according to some embodiments;

FIG. 4 is a perspective view of another example of a variable angle button-suture fixation assembly, according to some embodiments;

FIG. 5 is a sectional view of the variable angle button-suture fixation assembly of FIG. 4, according to some embodiments;

FIG. 6 is an exploded perspective view of the variable angle button-suture fixation assembly of FIG. 4, according to some embodiments;

FIG. 7 is a perspective view of another example of a variable angle button-suture fixation assembly, according to some embodiments;

FIG. 8 is a sectional view of the variable angle button-suture fixation assembly of FIG. 7, according to some embodiments;

FIG. 9 is an exploded perspective view of the variable angle button-suture fixation assembly of FIG. 7, according to some embodiments;

FIG. 10 is a perspective view of another example of a variable angle button-suture fixation assembly, according to some embodiments;

FIG. 11 is a sectional view of the variable angle button-suture fixation assembly of FIG. 10, according to some embodiments;

FIG. 12 is an exploded perspective view of the variable angle button-suture fixation assembly of FIG. 10, according to some embodiments;

FIG. 13 is a perspective view of another example of a variable angle button-suture fixation assembly, according to some embodiments;

FIG. 14 is a sectional view of the variable angle button-suture fixation assembly of FIG. 13, according to some embodiments;

FIG. 15 is an exploded perspective view of the variable angle button-suture fixation assembly of FIG. 13, according to some embodiments;

FIG. 16 is a perspective view of another example of a variable angle button-suture fixation assembly, according to some embodiments;

FIG. 17 is a sectional view of the variable angle button-suture fixation assembly of FIG. 16, according to some embodiments;

FIG. 18 is an exploded perspective view of the variable angle button-suture fixation assembly of FIG. 16, according to some embodiments;

FIG. 19 is a perspective view of another example of a variable angle button-suture fixation assembly, according to some embodiments;

FIG. 20 is a sectional view of the variable angle button-suture fixation assembly of FIG. 19, according to some embodiments;

FIG. 21 is an exploded perspective view of the variable angle button-suture fixation assembly of FIG. 19, according to some embodiments; and

FIGS. 22-24 are sectional views of a receiver forming part of a variable angle button-suture fixation assembly, illustrating various fixation angle ranges achievable by the variable angle button-suture assembly, according to some embodiments.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The variable angle button-suture fixation assembly and related methods disclosed herein boasts a variety of inventive features and components that warrant patent protection, both individually and in combination.

Described herein are several embodiments of a variable angle button-suture fixation assembly (or “variable angle button assembly” or “tensionable fixation assembly”) that enable suture assemblies to be deployed at more extreme angles, which are sometimes necessitated by the affected patient anatomy. The devices disclosed herein allow for the user to adjust the tension in a tensionable fixation member (e.g., surgical suture) at a more extreme angle than allowed by other systems (and also to adjust the angle of repair if needed during use), and then lock the construct with the tensionable fixation member under tension at the adjusted angle. Although described herein in the context of a knotless repair technique, the variable angle button-suture fixation assemblies described herein may be configured for use in a knotted fixation technique, or an interlocking suture fixation technique. By way of example, the knotless button-suture fixation technique referred to herein is generally shown and described in commonly-owned U.S. Pat. No. 11,109,855, issued Sep. 7, 2021, and entitled “Knotless Orthopedic Stabilization System” (“the '855 patent”), the entire contents of which is incorporated by reference as if set forth fully herein. Although shown and described herein as a surgical suture, it should be understood that the tensionable fixation member may be any form of flexible element capable of being locked under tension, including but not limited to surgical sutures, tapes, filaments, threads, wires, etc.

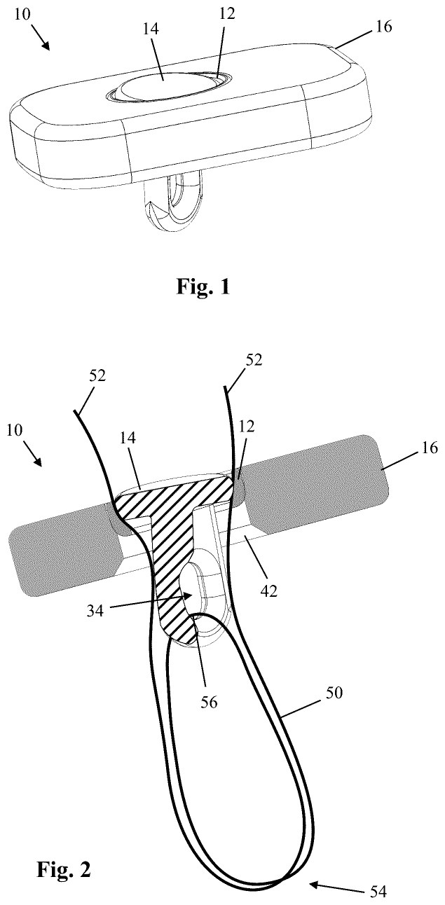

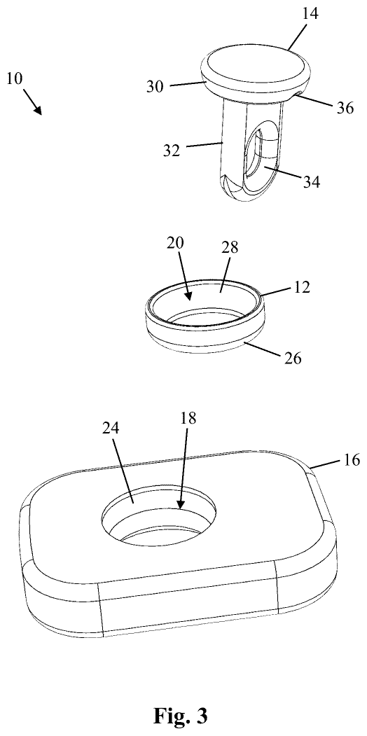

FIGS. 1-3 illustrate an example of a variable angle button assembly 10 according to one embodiment of the disclosure. In some embodiments, the variable angle button assembly 10 includes a button 12, a locking element 14, and a receiver 16. By way of example, the button 12 is received within an opening or through-hole 18 in the receiver 16, which for example may be an orthopedic fixation plate, another button, or a prosthesis, and the locking element 14 is received within an opening or through-hole 20 in the button 12. In this example embodiment, the opening 18 in the receiver 16 includes an inner recess 24 configured to receive the button 12 therein in such a way that the button 12 does not move relative to the receiver 16. In some embodiments, the receiver 16 further includes a chamfered, rounded, smooth, or angled surface 42 at the distal rim of the opening 18 to minimize the stress on the tensionable fixation member (e.g., surgical suture) 50 as it passes through and potentially contacts the receiver 16 during use, while enabling more extreme fixation angles.

In some embodiments, the button 12 has an outer circumferential surface 26 having a size and shape complementary to the size and shape of the inner recess 24 to facilitate the non-moving engagement between the button 12 and receiver 16. In some embodiments, the button 12 further includes an inner circumferential surface 28 configured to interface with the outer circumferential surface 30 of the locking element 14. In some embodiments, the inner circumferential surface 28 of the button 12 is concave and the outer circumferential surface 30 of the locking element 14 is convex, such that the locking element 14 is angularly moveable when seated within the button 12. Moreover, because of the interface between these complimentary curved surfaces, the locking element 14 may be secured within the button 12 at variable fixation angles while maintaining contact between the outer circumferential surface 30 of the locking element 14 and the inner circumferential surface 28 of the button 12, which ensures a lower profile of the button 12 even when fixation occurs at extreme angles. In some embodiments, the button 12 may have an upper or proximal surface that is chamfered to reduce or eliminate prominence of the button 12 beyond the proximal surface of the receiver 16.

In some embodiments, the locking element 14 further includes a distal protrusion 32 having one or more transverse openings 34 configured to receive one or more loops of a tensionable fixation member 50 therethrough. In some embodiments, the locking element 14 may have an upper or proximal surface that is chamfered to reduce or eliminate prominence of the locking element 14 beyond the proximal surface of the receiver 16.

In use, a portion of a tensionable fixation member 50 (e.g., one or more distal loop portions 54) is attached to or coupled with a tissue, bone, or other element while at least one other portion of the tensionable fixation member 50 (e.g., proximal loop portion 56) is looped through the transverse opening 34. One or more free ends 52 of the tensionable fixation member 50 pass between the button 12 and the locking element 14 within one or more lateral indentations 36 formed within the outer circumferential surface of the locking element 14. Prior to final tightening of the construct, the fixation angle of the locking element 14 relative to the button 12 (and receiver 16) may be established and/or adjusted to a desired or optimal degree to maintain tension in the tensionable fixation member 50. In some embodiments, the construct can then be locked with the desired tension in the tensionable fixation member 50 by exerting a proximal force (e.g., pulling) on the free ends 52 of the tensionable fixation member 50. This creates “pinch points” which capture and lock the tensionable fixation member 50 between the button 12 and locking element 14 within the lateral indentations 36 while maintaining the established or adjusted fixation angle of the locking element 14. In some embodiments, the construct can be locked while maintaining the fixation angle of the locking element 14 by tying one or more knots with the free ends 52 or by interweaving the free ends 52 in a finger-trap type design.

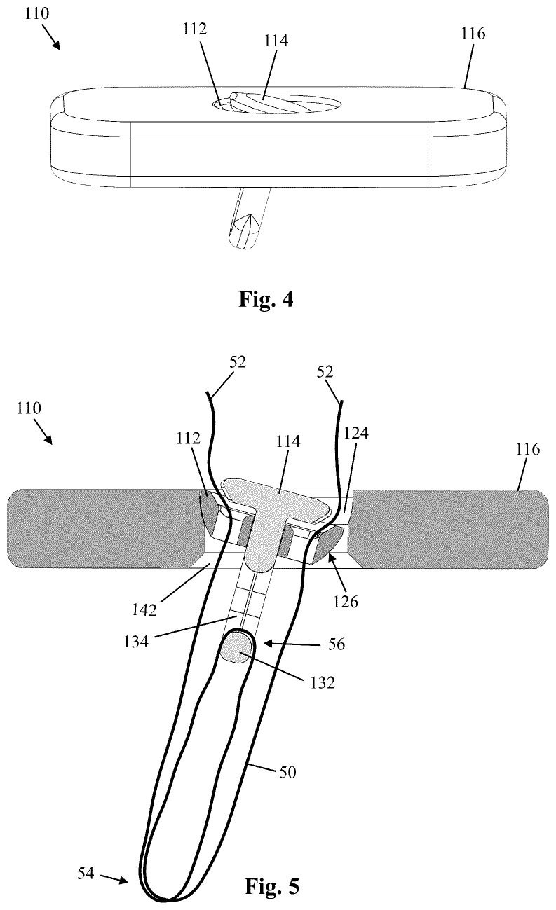

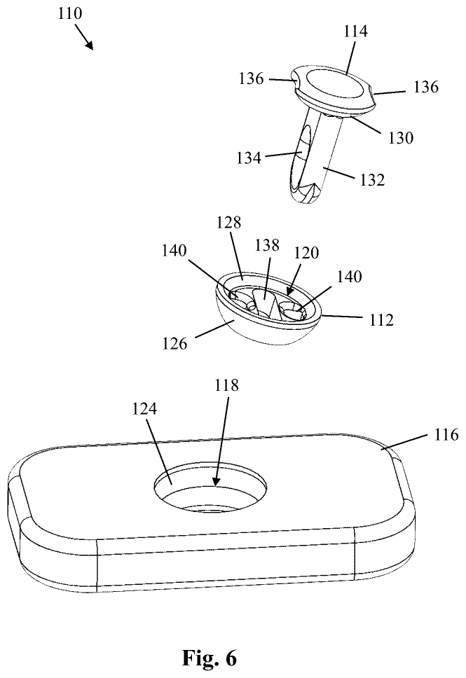

FIGS. 4-6 illustrate an example of a variable angle button assembly 110 according to one embodiment of the disclosure. In some embodiments, the variable angle button assembly 110 includes a button 112, a locking element 114, and a receiver 116. By way of example, the button 112 is received within an opening or through-hole 118 in the receiver 116, which for example may be an orthopedic fixation plate, and the locking element 114 is received within a recess 120 formed in a proximal surface of the button 112. In this example embodiment, the opening 118 in the receiver 116 includes an inner recess 124 configured to receive the button 112 therein in such a way that the button 112 is angularly moveable relative to the receiver 116 when the button 112 is seated within the inner recess 124. In some embodiments, the inner recess 124 comprises a concave circumferential surface configured to interface with a convex outer circumferential surface 126 of the button 112 such that the button 112 is angularly moveable when seated within the recess 124. Moreover, because of the interface between these complimentary curved surfaces, the button 112 may be secured within the recess 124 at variable fixation angles while maintaining contact between the outer circumferential surface 126 of the button 112 and the inner recess 124 of the receiver 116, which ensures a lower profile of the button 112 even when fixation occurs at extreme angles. In some embodiments, the receiver 116 further includes a chamfered, rounded, smooth, or angled surface 142 at the distal rim of the opening 118 to minimize the stress on the tensionable fixation member (e.g., surgical suture 50) as it passes through and potentially contacts the receiver 116 during use, while enabling more extreme fixation angles.

In some embodiments, the button 112 includes an inner circumferential surface 128 configured to interface with the outer circumferential surface 130 of the locking element 114 such that the locking element 114 does not angularly move relative to the button 112. In some embodiments, the inner circumferential surface 128 of the button 112 is conical and the outer circumferential surface 130 of the locking element 114 is also conical with a slope that matches the slope of the inner circumferential surface 128, such that the locking element 114 is angularly static when seated within the button 112. In some embodiments, the inner circumferential surface 128 of the button 112 and outer circumferential surface 130 of the locking element 114 may have complimentary surface structures (e.g., protrusion and recess) that facilitate a keyed engagement between the locking element 114 and button 112. In any event, the button 112 has an inner circumferential surface 128 having a size and shape complementary to the size and shape of the outer circumferential surface 130 of the locking element 114 to facilitate the non-moving engagement between the button 112 and locking element 114. In some embodiments, the locking element 114 further includes a distal protrusion 132 having one or more transverse openings 134 configured to receive one or more loops of a tensionable fixation member 50 therethrough.

In some embodiments, the button 112 includes a central opening 138 formed within the recess 120 and sized and configured to receive at least a portion of the distal protrusion 132 therethrough while preventing angular movement of the locking element 114 while the distal protrusion 132 is inserted into the central opening 138. In some embodiments, the button 112 includes one or more lateral openings 140 positioned within the recess on either side of the central opening 138 and configured to allow passage of the tensionable fixation member 50 therethrough. Although shown herein as having a pair of lateral openings 140, the button 112 may be configured with more or less than two lateral openings 140 without departing from the scope of the disclosure. In some embodiments, the central opening 138 may be formed perpendicular to a plane defined by the distal (e.g., bone or tissue engaging) surface of the receiver.

In some embodiments, the central opening 138 may be formed at an oblique angle relative to a plane defined by the distal (e.g., bone or tissue engaging) surface of the receiver 116. By way of example, this may include embodiments in which neither the button 112 nor the locking element 114 is angularly moveable relative to each other or the receiver 116. Thus, more extreme fixation angles may be achieved by providing a button 112 with the oblique angled central opening 138 and a locking element 114 with a complimentary oblique angled protrusion 132. While not a variable angle construct per se, a surgical kit may be provided with buttons 112 and locking elements 114 having several different fixation angles, e.g., 20°, 25°, 30°, 35° 40°, 45°, 50°, 55°, 60°, etc., to accommodate the needs of the individual surgeon at the time of surgery.

In some embodiments, the button 112 may have an upper or proximal surface that is chamfered to reduce or eliminate prominence of the button 112 beyond the proximal surface of the receiver 116. In some embodiments, the locking element 114 may have an upper or proximal surface that is chamfered to reduce or eliminate prominence of the locking element 114 beyond the proximal surface of the receiver 116.

In use, a portion of a tensionable fixation member 50 (e.g., one or more distal loop portions 54) is attached to or coupled with a tissue, bone, or other element while at least one other portion (e.g., one or more proximal loop portions 56) of the tensionable fixation member 50 is looped through the transverse opening 134. One or more free ends 52 of the tensionable fixation member 50 pass between the button 112 and the locking element 114 within one or more lateral indentations 136 formed within the outer circumferential surface 130 of the locking element 114. Prior to final tightening of the construct, the fixation angle of the button 112 relative to the receiver 116 may be established and/or adjusted to a desired or optimal degree to maintain tension in the tensionable fixation member 50. In some embodiments, the construct can then be locked with the desired tension in the tensionable fixation member 50 by exerting a proximal force (e.g., pulling) on the free ends 52 of the suture. This creates “pinch points” which capture and lock the tensionable fixation member 50 between the button 112 and locking element 114 within the lateral indentations 136 while maintaining the fixation angle of the button 112 relative to the receiver 116. In some embodiments, the construct can be locked while maintaining the fixation angle of the button 112 by tying one or more knots with the free ends 52 or by interweaving the free ends 52 in a finger-trap type design.

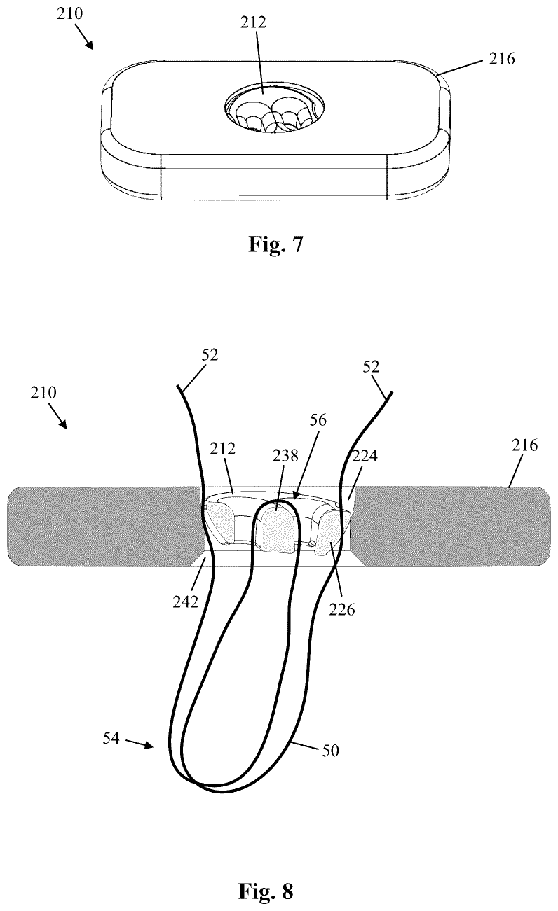

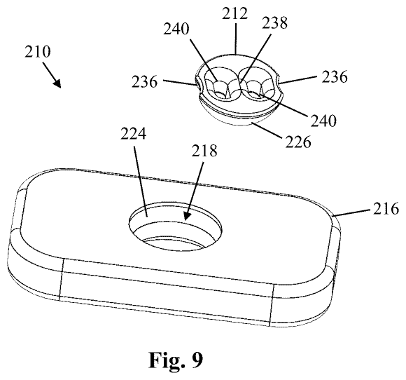

FIGS. 7-9 illustrate an example of a variable angle button assembly 210 according to one embodiment of the disclosure. By way of example, the variable angle button assembly 210 is similar to the button assemblies 10, 110 described above except that the assembly 210 does not include a separate locking element with a protrusion, which allow for even more extreme fixation angles. Instead, locking of the tensionable fixation member 50 occurs via interface between the button 212 and the receiver 216, as will be explained below. In some embodiments, the variable angle button assembly 210 includes a button 212 and a receiver 216. By way of example, the button 212 is received within an opening or through-hole 218 in the receiver 216, which for example may be an orthopedic fixation plate. In this example embodiment, the opening 218 in the receiver 216 includes an inner recess 224 configured to receive the button 212 therein in such a way that the button 212 is angularly moveable relative to the receiver 216 when the button 212 is seated within the inner recess 224. In some embodiments, the inner recess 224 comprises a concave circumferential surface configured to interface with a convex outer circumferential surface 226 of the button 212 such that the button 212 is angularly moveable when seated within the recess 224. Moreover, because of the interface between these complimentary curved surfaces, the button 212 may be secured within the recess 224 at variable fixation angles while maintaining contact between the outer circumferential surface 226 of the button 212 and the inner recess 224 of the receiver 216, which ensures a lower profile of the button 212 even when fixation occurs at extreme angles. In some embodiments, the receiver 216 further includes a chamfered, rounded, smooth, or angled surface 242 at the distal rim of the opening 218 to minimize the stress on the tensionable fixation member (e.g., surgical suture 50) as it passes through and potentially contacts the receiver 216 during use, while enabling more extreme fixation angles.

In some embodiments, the button 212 is round in shape (or oval, elliptical, or any geometric shape that allows for variable angle fixation) and includes a pair of lateral openings 240 positioned on either side of a central crossbar 238 and a pair of opposing lateral indentations 236 formed within the outer circumferential surface 226. The central crossbar 238 and lateral openings 240 enable a portion of the tensionable fixation member 50 to be looped over the crossbar 238 during use. Although shown herein as having a pair of lateral openings 240, the button 212 may be configured with more or less than two lateral openings 240 without departing from the scope of the disclosure.

In use, a portion of a tensionable fixation member 50 (e.g., a distal loop portion 54) is attached to or coupled with a tissue, bone, or other element while at least one other portion (e.g., a proximal loop portion 56) of the tensionable fixation member 50 is looped around the central crossbar 238 by way of the lateral openings 240. One or more free ends 52 of the tensionable fixation member 50 pass between the button 212 and the receiver 216 within the one or more lateral indentations 236 formed within the outer circumferential surface 226 of the button 212. Prior to final tightening of the construct, the fixation angle of the button 212 relative to the receiver 216 may be established and/or adjusted to a desired or optimal degree to maintain tension in the tensionable fixation member 50. In some embodiments, the construct can then be locked with the desired tension in the tensionable fixation member 50 by exerting a proximal force (e.g., pulling) on the free ends 52 of the tensionable fixation member 50. This creates “pinch points” which capture and lock the tensionable fixation member 50 between the button 212 and receiver 216 within the lateral indentations 236 while maintaining the fixation angle of the button 212 relative to the receiver 216. In some embodiments, the construct can be locked while maintaining the fixation angle of the button 212 by tying one or more knots with the free ends 52 or by interweaving the free ends 52 in a finger-trap type design.

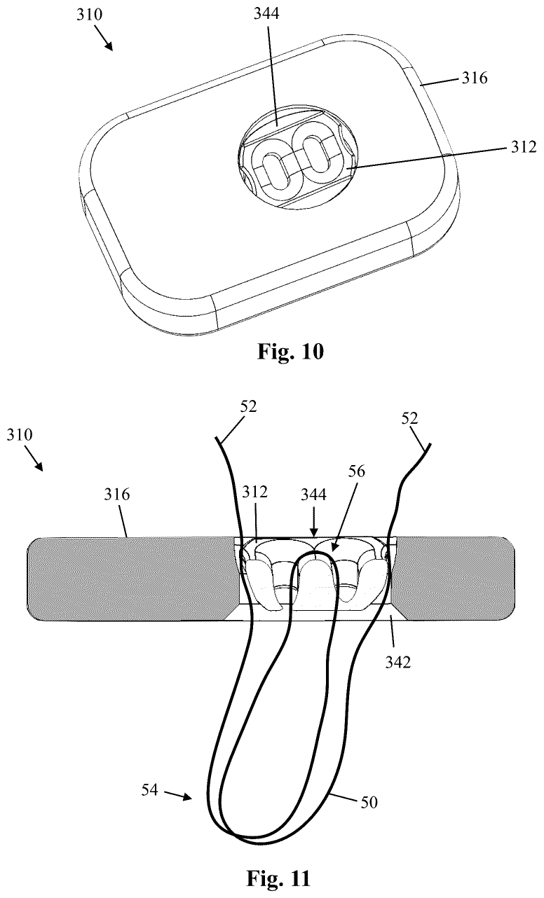

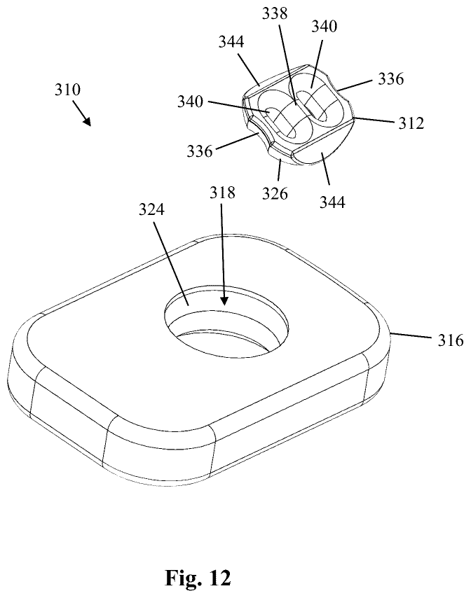

FIGS. 10-12 illustrate an example of a variable angle button assembly 310 according to one embodiment of the disclosure. By way of example, the variable angle button assembly 310 is similar to the button assemblies 10, 110 described above except that the assembly 310 does not include a separate locking element with a protrusion, which allow for even more extreme fixation angles. Instead, locking of the tensionable fixation member 50 occurs via interface between the button 312 and the receiver 316, as will be explained below. In some embodiments, the variable angle button assembly 310 includes a button 312 and a receiver 316. By way of example, the button 312 is received within an opening or through-hole 318 in the receiver 316, which for example may be an orthopedic fixation plate. In this example embodiment, the opening 318 in the receiver 316 includes an inner recess 324 configured to receive the button 312 therein in such a way that the button 312 is angularly moveable relative to the receiver 316 when the button 312 is seated within the inner recess 324. In some embodiments, the inner recess 324 comprises a concave circumferential surface configured to interface with a convex outer circumferential surface 326 of the button 312 such that the button 312 is angularly moveable when seated within the recess 324. Moreover, because of the interface between these complimentary curved surfaces, the button 312 may be secured within the recess 324 at variable fixation angles while maintaining contact between the outer circumferential surface 326 of the button 312 and the inner recess 324 of the receiver 316, which ensures a lower profile of the button 312 even when fixation occurs at extreme angles. In some embodiments, the receiver 316 further includes a chamfered, rounded, smooth, or angled surface 342 at the distal rim of the opening 318 to minimize the stress on the tensionable fixation member (e.g., surgical suture 50) as it passes through and potentially contacts the receiver 316 during use, while enabling more extreme fixation angles.

In some embodiments, the button 312 is round in shape (or oval, elliptical, or any geometric shape that allows for variable angle fixation) and includes a pair of lateral openings 340 positioned on either side of a central crossbar 338 and a pair of opposing lateral indentations 336 formed within the outer circumferential surface 326. The central crossbar 338 and lateral openings 340 enable a portion of the tensionable fixation member to be looped over the crossbar 338 during use. Although shown herein as having a pair of lateral openings 340, the button 312 may be configured with more or less than two lateral openings 340 without departing from the scope of the disclosure. In some embodiments, the proximal side of the button 312 may include angle surfaces or chamfers 344 to minimize or eliminate any portion of the button 312 that may otherwise protrude beyond the plane of the top or proximal surface 346 of the receiver 316, when the button 312 is oriented at an angle relative to the receiver 316.

In use, a portion of a tensionable fixation member 50 (e.g., a distal loop portion 54) is attached to or coupled with a tissue, bone, or other element while at least one other portion of the tensionable fixation member 50 (e.g., a proximal loop portion 56) is looped around the central crossbar 338 by way of the lateral openings 340. One or more free ends 52 of the tensionable fixation member 50 pass between the button 312 and the receiver 316 within the one or more lateral indentations 336 formed within the outer circumferential surface 326 of the button 312. Prior to final tightening of the construct, the fixation angle of the button 312 relative to the receiver 316 may be established and/or adjusted to a desired or optimal degree to maintain tension in the tensionable fixation member 50. In some embodiments, the construct can then be locked with the desired tension in the tensionable fixation member 50 by exerting a proximal force (e.g., pulling) on the free ends 52 of the suture. This creates “pinch points” which capture and lock the tensionable fixation member between the button 312 and receiver 316 within the lateral indentations 336 while maintaining the fixation angle of the button 312 relative to the receiver 316. In some embodiments, the construct can be locked while maintaining the fixation angle of the button 316 by tying one or more knots with the free ends 52 or by interweaving the free ends 52 in a finger-trap type design.

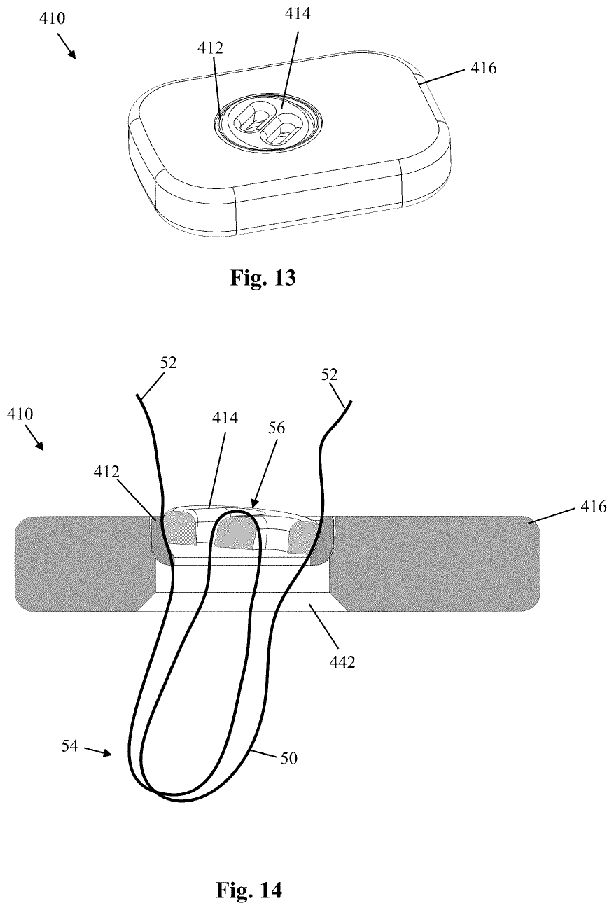

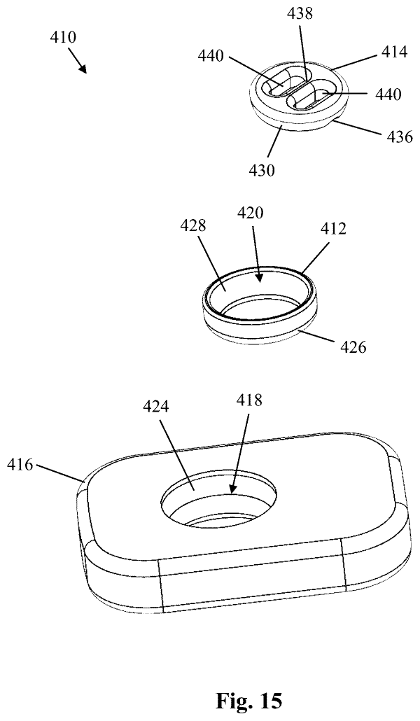

FIGS. 13-15 illustrate an example of a variable angle button assembly 410 according to one embodiment of the disclosure. In some embodiments, the variable angle button assembly 410 includes a button 412, a locking element 414, and a receiver 416. By way of example, the button 412 is received within an opening or through-hole 418 in the receiver 416, which for example may be an orthopedic fixation plate, and the locking element 414 is received within an opening or through-hole 420 in the button 412. In this example embodiment, the opening 418 in the receiver 416 includes an inner recess 424 configured to receive the button 412 therein in such a way that the button 412 does not move relative to the receiver 416. In some embodiments, the receiver 416 further includes a chamfered, rounded, smooth, or angled surface 442 at the distal rim of the opening 418 to minimize the stress on the tensionable fixation member (e.g., surgical suture) 50 as it passes through and potentially contacts the receiver 416 during use, while enabling more extreme fixation angles.

In some embodiments, the button 412 has an outer circumferential surface 426 having a size and shape complementary to the size and shape of the inner recess 424 to facilitate the non-moving engagement between the button 412 and receiver 416. In some embodiments, the button 412 further includes an inner circumferential surface 428 configured to interface with the outer circumferential surface 430 of the locking element 414. In some embodiments, the inner circumferential surface 428 of the button 412 is concave and the outer circumferential surface 430 of the locking element 414 is convex, such that the locking element 414 is angularly moveable when seated within the button 412. Moreover, because of the interface between these complimentary curved surfaces, the locking element 414 may be secured within the button 412 at variable fixation angles while maintaining contact between the outer circumferential surface 430 of the locking element 414 and the inner circumferential surface 428 of the button 412, which ensures a lower profile of the button 412 even when fixation occurs at extreme angles. In some embodiments, the button 412 may have an upper or proximal surface that is chamfered to reduce or eliminate prominence of the button 412 beyond the proximal surface of the receiver 416.

In some embodiments, the locking element 414 is round in shape (or oval, elliptical, or any geometric shape that allows for variable angle fixation) and includes a pair of lateral openings 440 positioned on either side of a central crossbar 438 and a pair of opposing lateral indentations 436 formed within the outer circumferential surface 430. The central crossbar 438 and lateral openings 440 enable a portion of the tensionable fixation member 50 to be looped over the crossbar 438 during use. Although shown herein as having a pair of lateral openings 440, the locking element 414 may be configured with more or less than two lateral openings 440 without departing from the scope of the disclosure. In some embodiments, the locking element 414 may have an upper or proximal surface that is chamfered to reduce or eliminate prominence of the locking element 414 beyond the proximal surface of the receiver 416.

In use, a portion of a tensionable fixation member 50 (e.g., a distal loop portion 54) is attached to or coupled with a tissue, bone, or other element while at least one other portion (e.g., a proximal loop portion 56) of the tensionable fixation member 50 is looped around the central crossbar 438 by way of the lateral openings 440. One or more free ends 52 of the tensionable fixation member 50 pass between the button 412 and the locking element 414 within one or more lateral indentations 436 formed within the outer circumferential surface 430 of the locking element 414. Prior to final tightening of the construct, the fixation angle of the locking element 414 relative to the button 412 (and receiver 416) may be established or adjusted to a desired or optimal degree to maintain tension in the tensionable fixation member 50. In some embodiments, the construct can then be locked with the desired tension in the tensionable fixation member 50 by exerting a proximal force (e.g., pulling) on the free ends 52 of the tensionable fixation member 50. This creates “pinch points” which capture and lock the tensionable fixation member 50 between the button 412 and locking element 414 within the lateral indentations 436 while maintaining the fixation angle of the locking element 414. In some embodiments, the construct can be locked while maintaining the fixation angle of the locking element 414 by tying one or more knots with the free ends 52 or by interweaving the free ends 52 in a finger-trap type design.

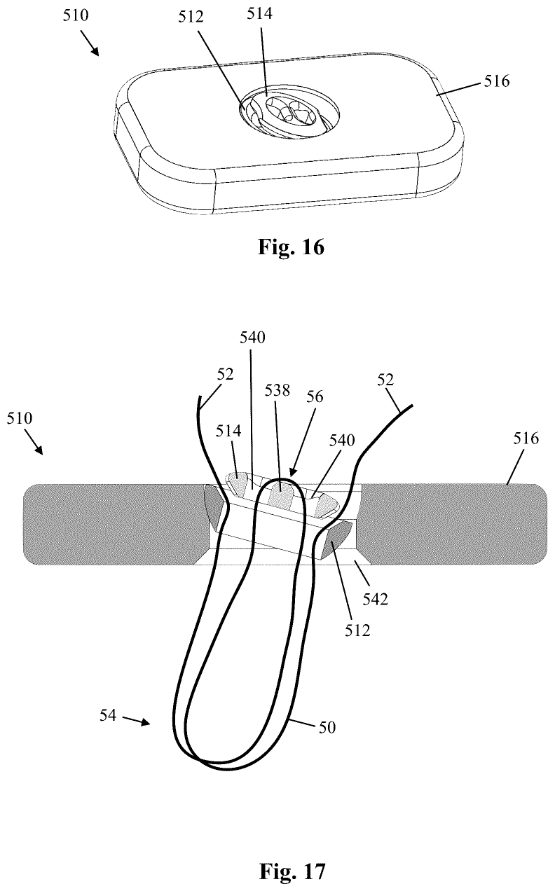

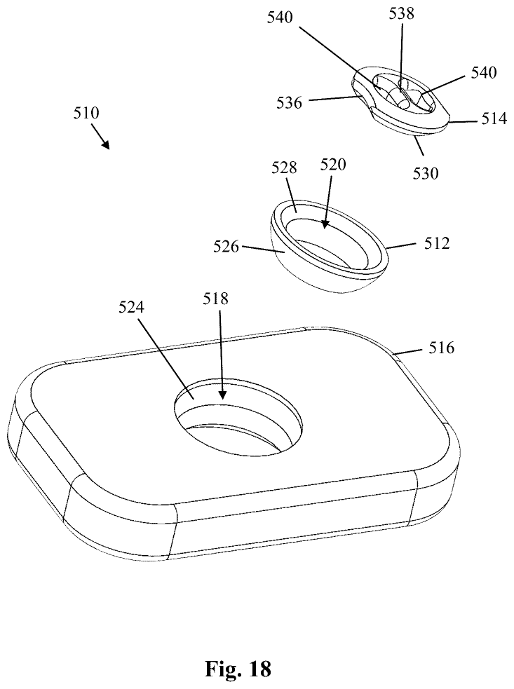

FIGS. 16-18 illustrate an example of a variable angle button assembly 510 according to one embodiment of the disclosure. In some embodiments, the variable angle button assembly 510 includes a button 512, a locking element 514, and a receiver 516. By way of example, the button 512 is received within an opening or through-hole 518 in the receiver 516, which for example may be an orthopedic fixation plate, and the locking element 514 is received within a recess 520 formed in a proximal surface of the button 512. In this example embodiment, the opening 518 in the receiver 516 includes an inner recess 524 configured to receive the button 512 therein in such a way that the button 512 is angularly moveable relative to the receiver 516 when the button 512 is seated within the inner recess 524. In some embodiments, the inner recess 524 comprises a concave circumferential surface configured to interface with a convex outer circumferential surface 526 of the button 512 such that the button 512 is angularly moveable when seated within the recess 524. Moreover, because of the interface between these complimentary curved surfaces, the button 512 may be secured within the recess 524 at variable fixation angles while maintaining contact between the outer circumferential surface 526 of the button 512 and the inner recess 524 of the receiver 516, which ensures a lower profile of the button 512 even when fixation occurs at extreme angles. In some embodiments, the receiver 516 further includes a chamfered, rounded, smooth, or angled surface 542 at the distal rim of the opening 518 to minimize the stress on the tensionable fixation member (e.g., surgical suture 50) as it passes through and potentially contacts the receiver 516 during use, while enabling more extreme fixation angles.

In some embodiments, the button 512 includes an inner circumferential surface 528 configured to interface with the outer circumferential surface 530 of the locking element 514 such that the locking element 514 does not angularly move relative to the button 512. In some embodiments, the inner circumferential surface 528 of the button 512 is conical and the outer circumferential surface 530 of the locking element 514 is also conical with a slope that matches the slope of the inner circumferential surface 528, such that the locking element 514 is angularly static when seated within the button 512. In some embodiments, the inner circumferential surface 528 of the button 512 and outer circumferential surface 530 of the locking element 514 may have complimentary surface structures (e.g., protrusion and recess) that facilitate a keyed engagement between the locking element 514 and button 512. In any event, the button 512 has an inner circumferential surface 528 having a size and shape complementary to the size and shape of the outer circumferential surface 530 of the locking element 514 to facilitate the non-moving engagement between the button 512 and locking element 514. In some embodiments, the button 512 may have an upper or proximal surface that is chamfered to reduce or eliminate prominence of the button 512 beyond the proximal surface of the receiver 516.

In some embodiments, the locking element 514 is round in shape (or oval, elliptical, or any geometric shape that allows for variable angle fixation) and includes a pair of lateral openings 540 positioned on either side of a central crossbar 538 and a pair of opposing lateral indentations 536 formed within the outer circumferential surface 530. The central crossbar 538 and lateral openings 540 enable a portion of the tensionable fixation member 50 to be looped over the crossbar 538 during use. Although shown herein as having a pair of lateral openings 540, the locking element 514 may be configured with more or less than two lateral openings 540 without departing from the scope of the disclosure. In some embodiments, the locking element 514 may have an upper or proximal surface that is chamfered to reduce or eliminate prominence of the locking element 514 beyond the proximal surface of the receiver 516.

In use, a portion of a tensionable fixation member 50 (e.g., a distal loop portion 54) is attached to or coupled with a tissue, bone, or other element while at least one other portion (e.g., a proximal loop portion 56) of the tensionable fixation member 50 is looped around the central crossbar 538 by way of the lateral openings 540. One or more free ends 52 of the tensionable fixation member 50 pass between the button 512 and the locking element 514 within one or more lateral indentations 536 formed within the outer circumferential surface 530 of the locking element 514. Prior to final tightening of the construct, the fixation angle of the button 512 relative to the receiver 516 may be established and/or adjusted to a desired or optimal degree to maintain tension in the tensionable fixation member 50. In some embodiments, the construct can then be locked with the desired tension in the tensionable fixation member 50 by exerting a proximal force (e.g., pulling) on the free ends 52 of the suture. This creates “pinch points” which capture and lock the tensionable fixation member 50 between the button 512 and locking element 514 within the lateral indentations 536 while maintaining the fixation angle of the button 512 relative to the receiver 516. In some embodiments, the construct can be locked while maintaining the fixation angle of the button 512 by tying one or more knots with the free ends 52 or by interweaving the free ends 52 in a finger-trap type design.

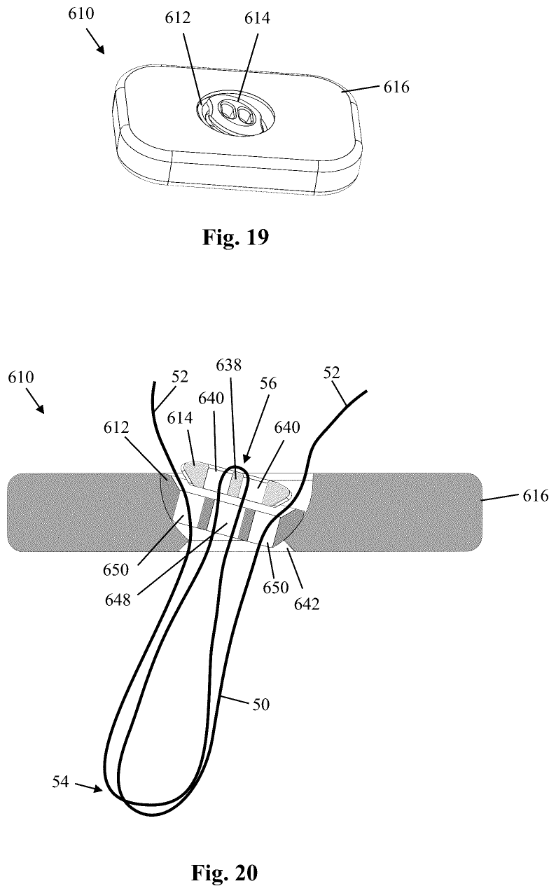

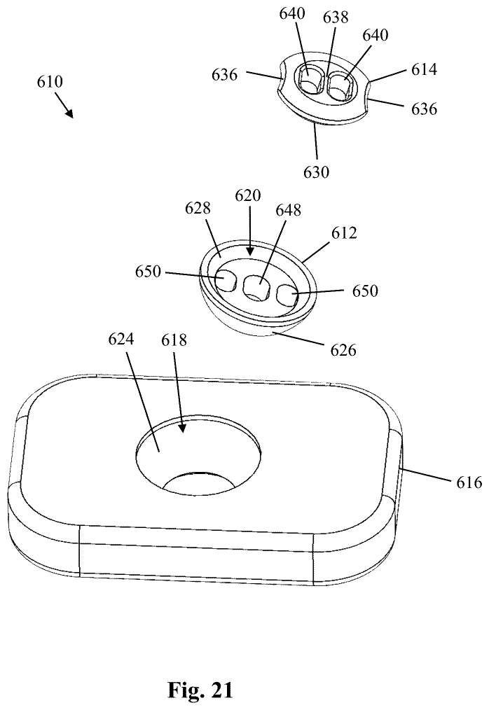

FIGS. 19-21 illustrate an example of a variable angle button assembly 610 according to one embodiment of the disclosure. In some embodiments, the variable angle button assembly 610 includes a button 612, a locking element 614, and a receiver 616. By way of example, the button 612 is received within an opening or through-hole 618 in the receiver 616, which for example may be an orthopedic fixation plate, and the locking element 614 is received within a recess 620 formed in a proximal surface of the button 612. In this example embodiment, the opening 618 in the receiver 616 includes an inner recess 624 configured to receive the button 612 therein in such a way that the button 612 is angularly moveable relative to the receiver 616 when the button 612 is seated within the inner recess 624. In some embodiments, the inner recess 624 comprises a concave circumferential surface configured to interface with a convex outer circumferential surface 626 of the button 612 such that the button 612 is angularly moveable when seated within the recess 624. Moreover, because of the interface between these complimentary curved surfaces, the button 612 may be secured within the recess 624 at variable fixation angles while maintaining contact between the outer circumferential surface 626 of the button 612 and the inner recess 624 of the receiver 616, which ensures a lower profile of the button 612 even when fixation occurs at extreme angles. In some embodiments, the receiver 616 further includes a chamfered, rounded, smooth, or angled surface 642 at the distal rim of the opening 618 to minimize the stress on the tensionable fixation member (e.g., surgical suture 50) as it passes through and potentially contacts the receiver 616 during use, while enabling more extreme fixation angles.

In some embodiments, the button 612 includes a central opening 648 formed within the recess 620 and sized and configured to enable passage of the tensionable fixation member 50 or other flexible fixation member therethrough. In some embodiments, the button 612 includes one or more lateral openings 650 positioned within the recess 620 on either side of the central opening 648 and configured to allow passage of the tensionable fixation member 50 therethrough. Although shown herein as having a pair of lateral openings 640, the button 612 may be configured with more or less than two lateral openings 640 without departing from the scope of the disclosure.

In some embodiments, the button 612 includes an inner circumferential surface 628 configured to interface with the outer circumferential surface 630 of the locking element 614 such that the locking element 614 does not angularly move relative to the button 612. In some embodiments, the inner circumferential surface 628 of the button 612 is conical and the outer circumferential surface 630 of the locking element 614 is also conical with a slope that matches the slope of the inner circumferential surface 628, such that the locking element 614 is angularly static when seated within the button 612. In some embodiments, the inner circumferential surface 628 of the button 612 and outer circumferential surface 630 of the locking element 614 may have complimentary surface structures (e.g., protrusion and recess) that facilitate a keyed engagement between the locking element 614 and button 612. In any event, the button 612 has an inner circumferential surface 628 having a size and shape complementary to the size and shape of the outer circumferential surface 630 of the locking element 614 to facilitate the non-moving engagement between the button 612 and locking element 614. In some embodiments, the button 612 may have an upper or proximal surface that is chamfered to reduce or eliminate prominence of the button 612 beyond the proximal surface of the receiver 616.

In some embodiments, the locking element 614 is round in shape (or oval, elliptical, or any geometric shape that allows for variable angle fixation) and includes a pair of lateral openings 640 positioned on either side of a central crossbar 638 and a pair of opposing lateral indentations 636 formed within the outer circumferential surface 630. The central crossbar 638 and lateral openings 640 enable a portion of the tensionable fixation member 50 to be looped over the crossbar 638 during use. Although shown herein as having a pair of lateral openings 640, the locking element 614 may be configured with more or less than two lateral openings 640 without departing from the scope of the disclosure. In some embodiments, the locking element 614 may have an upper or proximal surface that is chamfered to reduce or eliminate prominence of the locking element 614 beyond the proximal surface of the receiver 616.

In use, a portion of a tensionable fixation member 50 (e.g., a distal loop portion 54) is attached to or coupled with a tissue, bone, or other element while at least one other portion (e.g., a proximal loop portion 56) of the tensionable fixation member 50 is looped around the central crossbar 638 by way of the lateral openings 640 of the locking element 614 and the central opening 648 of the button 612. One or more free ends 52 of the tensionable fixation member 50 pass through lateral openings 650 of the button 612 and then pass between the button 612 and the locking element 614 within one or more lateral indentations 636 formed within the outer circumferential surface 630 of the locking element 614. Prior to final tightening of the construct, the fixation angle of the button 612 relative to the receiver 616 may be established and/or adjusted to a desired or optimal degree to maintain tension in the tensionable fixation member 50. In some embodiments, the construct can then be locked with the desired tension in the tensionable fixation member 50 by exerting a proximal force (e.g., pulling) on the free ends 52 of the suture. This creates “pinch points” which capture and lock the tensionable fixation member 50 between the button 612 and locking element 614 within the lateral indentations 636 while maintaining the fixation angle of the button 612 relative to the receiver 616. In some embodiments, the construct can be locked while maintaining the fixation angle of the button 612 by tying one or more knots with the free ends 52 or by interweaving the free ends 52 in a finger-trap type design.

Although shown and described herein as a fixation plate, it should be understood that the receivers 16, 116, 216, 316, 416, 516, 616 described herein may be any device that is capable or for has been adapted to receive the button 12, 112, 212, 312, 412, 512, 612 therein, including but not limited to a fixation plate, button, prosthesis, etc. Moreover, the receiver may include multiple openings for use with multiple variable angle buttons and/or non variable angle buttons and/or bone screws to achieve fixation. In some embodiments, the device 110, 210, 310 can be used without a receiver member, or stated another way, the receiver and button may be interchangeable elements such that the device may be placed directly on the bone without a fixation plate or other intermediary device.

By way of example, the buttons 112, 212, 312, 512, 612 and/or locking elements 14, 414 described herein may be adjusted to any angle (e.g., “adjustable angle” or “fixation angle” A₁ measured between a longitudinal axis L₁ of the opening or through-hole 18, 118, 218, 318, 418, 518, 618 of the receiver 16, 116, 216, 316, 416, 516, 616 and a longitudinal axis L2 of the button 112, 212, 312, 512, 612) up to a maximum angle that is allowable by the physical constraints of the receiver. By way of example, when the button 112, 212, 312, 512, 612 is seated in the receiver 116, 216, 316, 516, 616 in an angularly neutral position, the longitudinal axis L₁ of the button 112, 212, 312, 512, 612 and the longitudinal axis L₂ of the receiver 116, 216, 316, 516, 616 are in alignment, with 0° of angulation between them. As used herein, the term “angularly moveable” means that the button 112, 212, 312, 512, 612 (prior to final tightening) may move (e.g., pivot, rotate, etc.) within the inner recess 124, 224, 324, 524, 624 of the receiver 116, 216, 316, 516, 616 to any adjusted angle or fixation angle where the angle A₁ between the longitudinal axis L₁ of the receiver 116, 216, 316, 516, 616 and the longitudinal axis L₂ of the button 112, 212, 316, 512, 612 is nonzero while maintaining contact between the circumferential mating surface 126, 226, 326, 526, 626 of the button and the inner recess 124, 224, 324, 524, 624 of the receiver 116, 216, 316, 516, 616.

Similarly, when the locking element 14, 414 is seated in the button 12, 412, at an angularly neutral position, the longitudinal axis L₁ of the button 12, 412 and the longitudinal axis L₂ of the locking element 14, 414 are in alignment, with 0° of angulation between them. As used herein, the term “angularly moveable” means that the locking element 14, 414 (prior to final tightening) may move (e.g., pivot, rotate, etc.) within the inner recess 28, 428 of the button 12, 412 to any adjusted angle or fixation angle where the angle A₁ between the longitudinal axis L₁ of the button 12, 412 and the longitudinal axis L₂ of the locking element 14, 414 is nonzero while maintaining contact between the mating surface of the locking element 30, 430 and the receiving surface 28, 428 of the button.

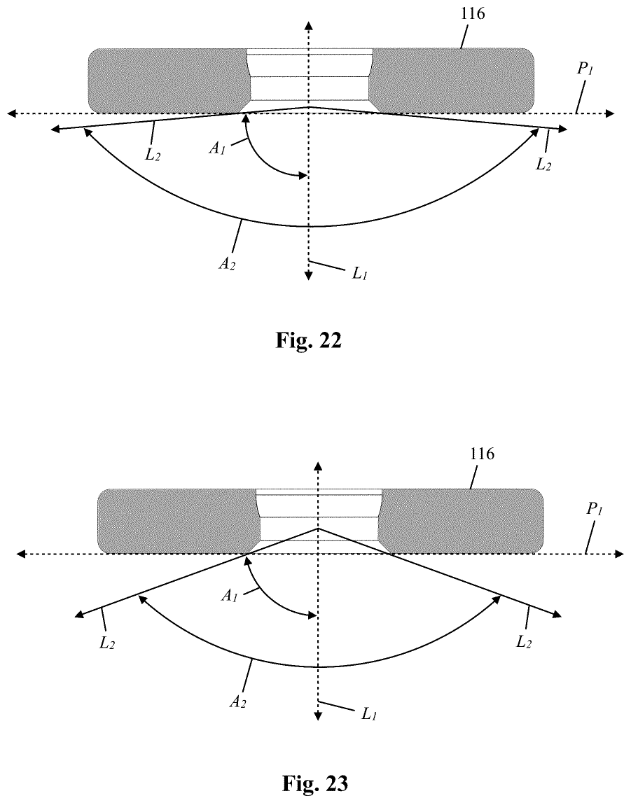

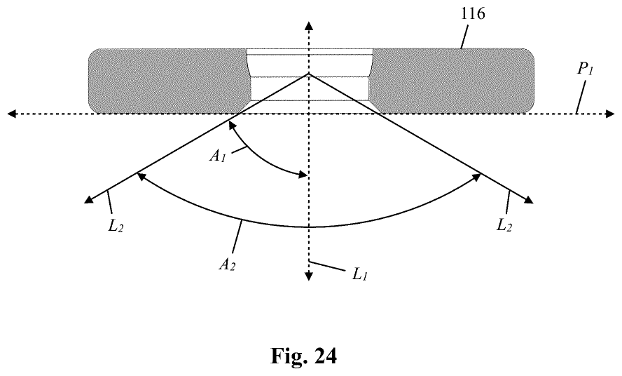

In some embodiments, the fixation angle A₁ may be within a range between 1° to 85°, which results in a total angular range A₂ of between 5° and 175° relative to the plane P₁ defined by the distal surface of the receiver 116, as illustrated by way of example only in FIG. 22. In some embodiments, the fixation angle A, may be within a range between 1° and 70°, which results in a total angular range A₂ between 20° and 160°, as shown by way of example only in FIG. 23. In some embodiments, the fixation angle A₁ may be within a range between 1° and 60°, which results in a total angular range A₂ of 30-150°, as shown by way of example only in FIG. 24.

In some embodiments, the button, locking element, and/or receiver may be manufactured from any suitable surgical grade material, including but not limited to metal (e.g., titanium), plastics (e.g., PEEK), or other materials.

From the foregoing disclosure and detailed description of certain preferred embodiments, it is also apparent that various modifications, additions, and other alternative embodiments are possible without departing from the true scope and spirit. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

What is claimed is:

1. A tensionable fixation assembly configured to fix tissue or bone to tissue, bone, or other member, comprising:
a button comprising a proximal facing surface, a distal facing surface, a circumferential outer mating surface, and at least one opening extending therethrough, the button defining a first end of the tensionable fixation assembly;
a receiver having an opening extending therethrough and an inner recess surrounding the opening, the inner recess defining a receiving surface configured to interface with the button such that the button is angularly moveable relative to the receiver and configured to be secured within the recess at variable fixation angles while maintaining contact between the outer mating surface and the receiving surface; and
at least one tensionable fixation member interacting with the button and receiver.

2. The tensionable fixation assembly of claim 1, wherein the at least one tensionable fixation member has a proximal end portion extending proximally from the button and a first loop portion extending distally from the receiver and configured to mate with tissue, bone, or other member, the first loop portion defining a second end of the tensionable fixation assembly.

3. The tensionable fixation assembly of claim 2, wherein the tensionable fixation assembly comprises a first state defining a first distance between the first and second ends and second state defining a second distance between the first and second ends, the second distance being less than the first distance.

4. The tensionable fixation assembly of claim 3, wherein the at least one tensionable fixation member passes through the button and receiver.

5. The tensionable fixation assembly of claim 4, wherein the at least one tensionable fixation member passes through the button and receiver by passing between the receiving surface of the receiver and the mating surface of the button.

6. The tensionable fixation assembly of claim 5, wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained in a knotless manner via compression and friction applied to the tensionable fixation member between the receiving surface of the receiver and the mating surface of the button while maintaining the fixation angle of the button relative to the receiver.

7. The tensionable fixation assembly of claim 4, wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained by tying one or more knots in the proximal end portion of the tensionable fixation member while maintaining the fixation angle of the button relative to the receiver.

8. The tensionable fixation assembly of claim 4, wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained by interweaving the proximal end portion of the tensionable fixation member while maintaining the fixation angle of the button relative to the receiver.

9. The tensionable fixation assembly of claim 8, wherein the interweaving comprises a finger trap design.

10. The tensionable fixation assembly of claim 1, wherein the receiver comprises an orthopedic fixation plate, a second button, or a prosthesis.

11. The tensionable fixation assembly of claim 1, wherein the button comprises at least one chamfered proximal surface.

12. A tensionable fixation assembly configured to fix tissue or bone to tissue, bone, or other member, comprising:
a suspension device defining a first end and comprising a button and a locking element separable from the button, the button having a proximal facing surface, a distal facing surface, a circumferential outer mating surface, a proximal recess including a receiving surface configured to mate with the locking element, and at least one opening extending therethrough, the locking element having a mating surface configured to interface with the receiving surface of the button;
a receiver having an opening extending therethrough and an inner recess surrounding the opening, the inner recess defining a receiving surface configured to interface with the button such that the button is angularly moveable relative to the receiver and configured to be secured within the recess at variable fixation angles while maintaining contact between the outer mating surface and the receiving surface;
at least one tensionable fixation member interacting with the suspension device, the at least one tensionable fixation member having a proximal end portion extending proximally from the suspension device, the at least one tensionable fixation member passing through the suspension device and forming a first loop portion extending distally from the suspension device, the first loop portion defining a second end configured to mate with tissue, bone, or other member, the at least one tensionable fixation member passing through the suspension device between the receiving surface of the button and the mating surface of the locking element;
wherein the tensionable fixation assembly comprises a first state defining a first distance between the first and second ends and second state defining a second distance between the first and second ends, the second distance being less than the first distance;
wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained via compression and friction applied to the tensionable fixation member between the receiving surface of the button and the mating surface of the locking pin while maintaining the fixation angle of the button relative to the receiver.

13. The tensionable fixation assembly of claim 12, wherein the mating surface of the locking element and the receiving surface of the button are configured to interface with each other such that the locking element does not angularly move relative to the button.

14. The tensionable fixation assembly of claim 13, wherein the receiving surface of the button is conical, and the mating surface of the locking element is conical.

15. The tensionable fixation assembly of claim 12, wherein the inner recess of the receiver comprises a concave circumferential surface and the outer mating surface of the button comprises a convex outer circumferential surface.

16. The tensionable fixation assembly of claim 12, wherein the receiver comprises an orthopedic fixation plate, a second button, or a prosthesis.

17. The tensionable fixation assembly of claim 12, wherein the fixation angle is within the range of between 1° and 60°.

18. The tensionable fixation assembly of claim 12, wherein the fixation angle is within the range of between 1° and 70°.

19. The tensionable fixation assembly of claim 12, wherein the fixation angle is within the range of between 1° and 85°.

20. A tensionable fixation assembly configured to fix tissue or bone to tissue, bone, or other member, comprising:
a suspension device defining a first end and comprising a button and a locking element separable from the button, the button having a proximal facing surface, a distal facing surface, a circumferential outer mating surface, a proximal recess including a receiving surface configured to mate with the locking element, and at least one opening extending therethrough, the locking element having a mating surface configured to mate with the receiving surface of the button such that the locking element is angularly moveable relative to the button and configured to be secured within the proximal recess at variable fixation angles while maintaining contact between the mating surface of the locking element and the receiving surface of the button;
a receiver having an opening extending therethrough and an inner recess surrounding the opening and configured to receive the button therein such that the button is not angularly moveable relative to the receiver when the button is seated within the inner recess;
at least one tensionable fixation member interacting with the suspension device, the at least one tensionable fixation member having a proximal end portion extending proximally from the suspension device, the at least one tensionable fixation member passing through the suspension device and forming a first loop portion extending distally from the suspension device, the first loop portion defining a second end configured to mate with tissue, bone, or other member, the at least one tensionable fixation member passing through the suspension device between the receiving surface of the button and the mating surface of the locking element;
wherein the tensionable fixation assembly comprises a first state defining a first distance between the first and second ends and second state defining a second distance between the first and second ends, the second distance being less than the first distance;
wherein the proximal end portion of the tensionable fixation member is configured to be pulled taut in the proximal direction when the first end of the tensionable fixation assembly is mated with a first tissue or bone and the second end of the tensionable fixation assembly is mated with a second tissue, bone, or other member, thereby creating tension in the tensionable fixation member which causes the tensionable fixation assembly to transition from the first state to the second state, where the second state is maintained via compression and friction applied to the tensionable fixation member between the receiving surface of the button and the mating surface of the locking element while maintaining the fixation angle of the locking element relative to the button.

21. The tensionable fixation assembly of claim 20, wherein the inner recess of the button comprises a concave circumferential surface and the outer mating surface of the locking element comprises a convex outer circumferential surface.

22. The tensionable fixation assembly of claim 20, wherein the button comprises at least one chamfered proximal surface.

23. The tensionable fixation assembly of claim 20, wherein the receiver comprises an orthopedic fixation plate, a second button, or a prosthesis.

24. The tensionable fixation assembly of claim 20, wherein the fixation angle is within the range of between 1° and 60°.

25. The tensionable fixation assembly of claim 20, wherein the fixation angle is within the range of between 1° and 70°.

26. The tensionable fixation assembly of claim 20, wherein the fixation angle is within the range of between 1° and 85°.
